(19) United States
(12) Patent Application Publication
(10) Pub. No.: US 2022/0017552 A1
(43) Pub. Date: Jan. 20, 2022

(54) ELECTROLUMINESECENT MATERIAL, METHOD FOR MANUFACTRUING THE SAME, AND LUMINESECENT DEVICE

(71) Applicant: Wuhan China Stra Optoelectronics Semiconductor Display Technology Co., LTD., Wuhan, Hubei (CN)

(72) Inventors: Jiajia LUO, Wuhan, Hubei (CN); Xianjie LI, Wuhan, Hubei (CN); Yu GU, Wuhan, Hubei (CN); Jinchang HUANG, Wuhan, Hubei (CN); Lin YANG, Wuhan, Hubei (CN); Yamei BAI, Wuhan, Hubei (CN)

(21) Appl. No.: 16/319,881

(22) PCT Filed: Dec. 24, 2018
(86) PCT No.: PCT/CN2018/123169

§ 371 Date: Jan. 23, 2019

(30) Foreign Application Priority Data
Oct. 17, 2018 (CN) 201811208818.7

(51) Int. Cl. C07F 9/92; C09K 11/06; H01L 51/00

(52) U.S. Cl. CPC C07F 9/92; C09K 11/06; H01L 51/5016; H01L 2251/552; H01L 51/0072

(57) ABSTRACT

The present application provides a luminescent material, a method for manufacturing the luminescent material and a luminescent device, the method includes providing a first reactant and a second reactant, reacting the first reactant and the second reactant to generate a mixture containing the luminescent material, separating and purifying the mixture containing the luminescent material to obtain the luminescent material.

### CROSS REFERENCE TO RELATED APPLICATION

This application claims the priority of International Application No. PCT/CN2018/123169, filed on 2018 Dec. 24, which claims priority to Chinese Application No. 201811208818.7, filed on 2018 Dec. 17. The entire disclosures of each of the above applications are incorporated herein by reference.

### BACKGROUND OF INVENTION

### Field of Invention

The present invention relates to a lighting field, and particularly to an electroluminescent material, a method for manufacturing the electroluminescent material, and a luminescent device.

### Description of Prior Art

In prior art, organic light emitting diode (OLED) has a self-luminous property, and a dominant light emitting material is mainly an electroluminescent material. However, lifespan of a blue light emitting electroluminescent material is short and causes failure of OLED, therefore, it is necessary to provide a blue light emitting electroluminescent material with a long lifespan, a method of manufacturing the electroluminescent material, and a luminescent device.

### SUMMARY OF INVENTION

An electroluminescent material and a method for manufacturing the electroluminescent material, and a luminescent device are provided to realize a blue light emitting electroluminescent material with a long lifespan, a method of manufacturing the electroluminescent material, and a luminescent device.

An electroluminescent material, wherein a molecular structural formula of the electroluminescent material is as follows:

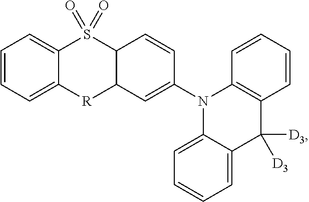

wherein R is

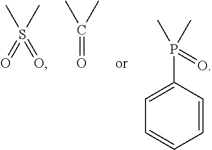

In the electroluminescent material of the present disclosure, an emission wavelength of the electroluminescent material ranges from 425 nanometers to 475 nanometers.

In the electroluminescent material of the present disclosure, the R is

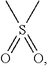

a molecular structural formula of the electroluminescent material is

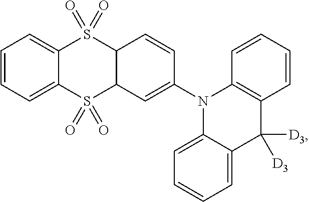

an emission wavelength of the electroluminescent material with the molecular structural formula

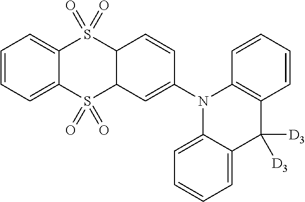

ranges from 425 nanometers to 450 nanometers.

In the electroluminescent material of the present disclosure, the R is

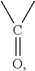

a molecular structural formula of the electroluminescent material is

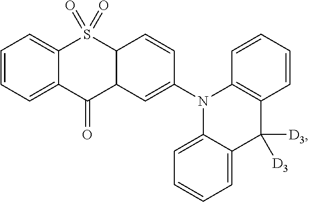

an emission wavelength of the electroluminescent material with the molecular structural formula

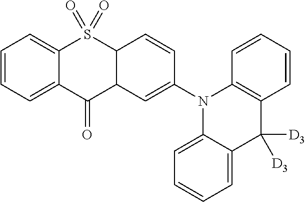

ranges from 430 nanometers to 460 nanometers.

In the electroluminescent material of the present disclosure, the R is

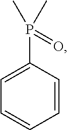

a molecular structural formula of the electroluminescent material is

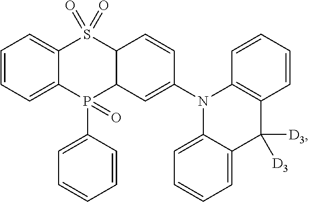

an emission wavelength of the electroluminescent material with the molecular structural formula

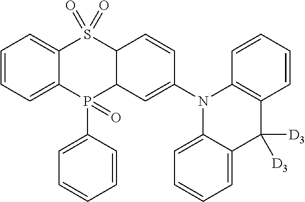

ranges from 450 nanometers to 475 nanometers.

A method for manufacturing an electroluminescent material includes:

providing a first reactant and a second reactant, reacting the first reactant and the second reactant to generate the electroluminescent material, a molecular structural formula of the first reactant is

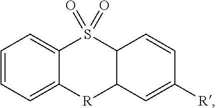

### and the R is

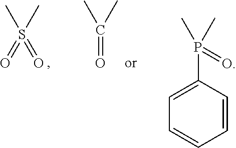

the R′ is Cl, Br, or I, the second reactant is 9, 10-dihydrogen-9, 9-dideuterium methyl acridine, and a molecular structural formula of the second reactant is

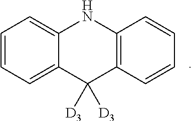

In the method for manufacturing the electroluminescent material of the present disclosure, a mole ratio of the first reactant and the second reactant ranges from 1:0.5 to 1:2.

In the method for manufacturing the electroluminescent material of the present disclosure, the first reactant and the second reactant are reacted in a solvent to generate the electroluminescent material, and the solvent is selected from a group consisting of styrene, perchloroethylene, methylbenzene, trichloroethylene, acetone, ethylene glycol ether, and triethanolamine.

In the method for manufacturing the electroluminescent material of the present disclosure, the solvent includes an additive, and the additive is selected from a group consisting of potassium hydroxide, sodium hydroxide, sodium hydrogencarbonate, [1,1′-bis(diphenylphosphino)ferrocene]palladium dichloride (Pd(dppf)Cl₂), potassium acetate (KOAc), sodium acetate (NaOAc), potassium nitrate (KNO₃), palladium acetate (Pd(OAc)₂), magnesium sulfate (MgSO₄), sodium t-butoxide (NaOt-Bu) and tri-tert-butylphosphine tetrafluoroborate.

In the method for manufacturing the electroluminescent material of the present disclosure, a reaction temperature of the first reactant and the second reactant reacted to generate the electroluminescent material ranges from 100 degrees centigrade to 150 degrees centigrade.

In the method for manufacturing the electroluminescent material of the present disclosure, a reaction time of the first reactant and the second reactant reacted to generate the electroluminescent material ranges from 12 hours to 64 hours.

In the method for manufacturing the electroluminescent material of the present disclosure, a molecular structural formula of the electroluminescent material is as follows:

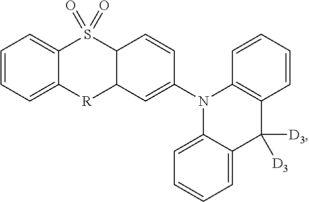

wherein R is

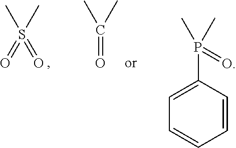

In the method for manufacturing the electroluminescent material of the present disclosure, the step of providing the first reactant and the second reactant, the first reactant and the second reactant are reacted to generate the electroluminescent material includes:

providing a first reactant and a second reactant;

reacting the first reactant and the second reactant to generate a mixture containing the electroluminescent material; and

separating and purifying the mixture containing the electroluminescent material to obtain the electroluminescent material.

In the method for manufacturing the electroluminescent material of the present disclosure, the step of separating and purifying the mixture containing the electroluminescent material to obtain the electroluminescent material includes:

extracting the mixture containing the electroluminescent material by an extraction solvent; and employing column chromatography to the mixture containing the electroluminescent material by a chromatographic column.

In the method for manufacturing the electroluminescent material of the present disclosure, the extraction solvent is selected from a group consisting of dichloromethane, trichloromethane and tetrahydrofuran.

In the method for manufacturing the electroluminescent material of the present disclosure, a volume ratio of the dichloromethane and n-hexane of the chromatographiccolum ranges from 1:0.5 to 1:10.

A luminescent device includes:

a base layer, the base layer includes a substrate base and a first electrode layer formed on the substrate base;

a cavity transmitting and injecting layer, the cavity transmitting and injecting layer is formed on the base layer, and electrically connected to the first electrode layer;

a luminescent layer, the luminescent layer is formed on the cavity transmitting and injecting layer;

an electronic transmitting layer, the electronic transmitting layer is formed on the luminescent layer; and

a second electrode layer, the second electrode layer is electrically connected to the electronic transmitting layer, wherein the luminescent layer includes a electroluminescent material, a molecular structural formula of the electroluminescent material is:

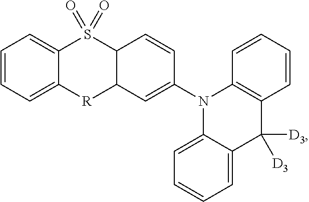

wherein the R is

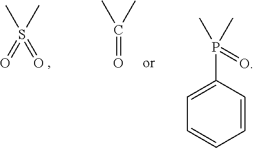

In the luminescent device of the present disclosure, the R is

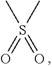

a molecular structural formula of the electroluminescent material is

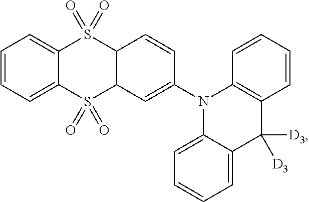

an emission wavelength of the electroluminescent material with the molecular structural formula

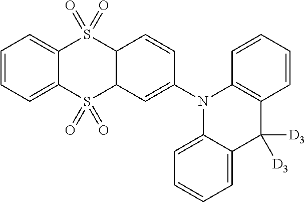

ranges from 425 nanometers to 450 nanometers.

In the electroluminescent material of the present disclosure, the R is

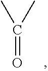

a molecular structural formula of the electroluminescent material is

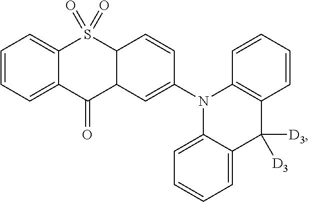

an emission wavelength of the electroluminescent material with the molecular structural formula

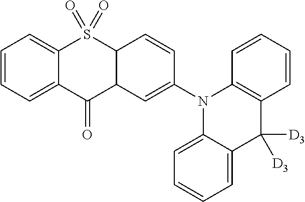

ranges from 430 nanometers to 460 nanometers.

In the electroluminescent material of present disclosure, the R is

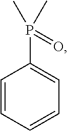

a molecular structural formula of the electroluminescent material is

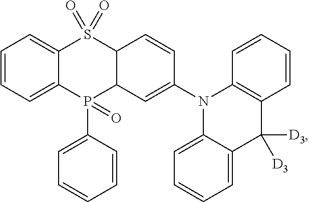

an emission wavelength of the electroluminescent material with the molecular structural formula

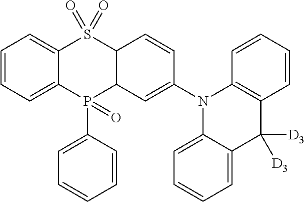

been ranges from 450 nanometers to 475 nanometers.

The benefit of the present disclosure is: an electroluminescent material, a method for manufacturing the electroluminescent material and luminescent device are provided and include: providing a first reactant and a second reactant, reacting the first reactant and the second reactant to generate a mixture containing the electroluminescent material, and a molecular structural formula of the first reactant is

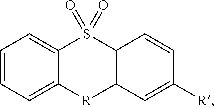

### and the R is

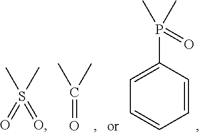

the R′ is Cl, Br, or I, the second reactant is 9, 10-dihydrogen-9, 9-dideuterium methyl acridine, a molecular structural formula of the second reactant is

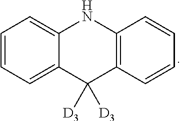

separating and purifying the mixture containing the electroluminescent material to obtain the electroluminescent material to realize a blue light emitting electroluminescent material with a long lifespan, and a method for manufacturing the electroluminescent material, and a luminescent device.

### BRIEF DESCRIPTION OF DRAWINGS

In order to explain more clearly the embodiments in the present application or the technical solutions in prior art, the following will briefly introduce the figures needed in the description of the embodiments or prior art. Obviously, figures in the following description are only some embodiments recorded in the present application, and for persons skilled in the art, other figures may also be obtained based on these figures without paying creative efforts.

FIG. 1 is a photoluminescence spectrum of an electroluminescent material been in toluene of the present application.

FIG. 2 is a structural view of a luminescent device of the present application.

FIG. 3 is a comparison chart of a luminous efficiency attenuated by time of luminescent devices of prior art and the present application.

### DETAILED DESCRIPTION OF PREFERRED EMBODIMENTS

In order to enable the persons skilled in the art to better understand the technical solutions in this application, clear and comprehensive description will be made to the technical solutions in the embodiments of the present application will be made in the following in combination with the figures in the embodiments of the present application. Obviously, the embodiments described herein are only part of, rather than all of, the embodiments of the present application. Based on the embodiments of the present application, all other embodiments obtained by ordinary skilled persons in the field without paying creative efforts should pertain to the extent of protection of the present application.

The present application provides an electroluminescent material. A molecular structural formula of the electroluminescent material is as follows:

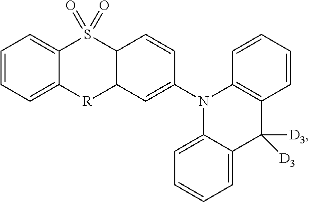

wherein the R is

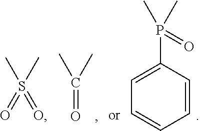

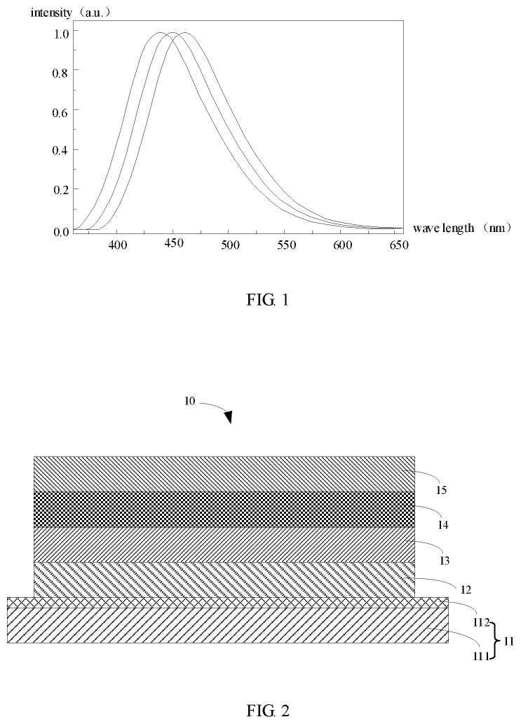

Referring to FIG. 1, FIG. 1 is a photoluminescence spectrum of an electroluminescent material been in toluene of the present application. An emission wavelength of the electroluminescent material ranges from 425 nanometers to 475 nanometers. Light emitted by the electroluminescent material is blue light.

In one embodiment, when the R is

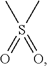

a molecular structural formula of an electroluminescent material is

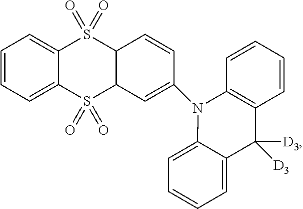

an emission wavelength of the electroluminescent material with the molecular structural formula

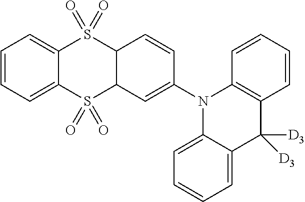

ranges from 425 nanometers to 450 nanometers.

In one embodiment, when the R is

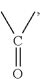

a molecular structural formula of an electroluminescent material is

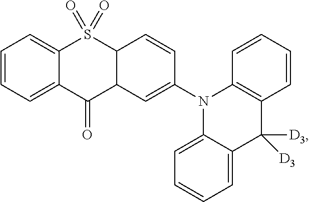

an emission wavelength of the electroluminescent material with the molecular structural formula

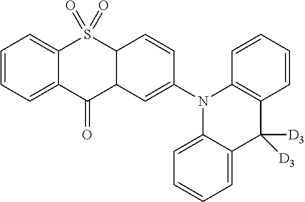

ranges from 430 nanometers to 460 nanometers.

In one embodiment, when the R is

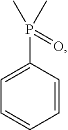

a molecular structural formula of an electroluminescent material is

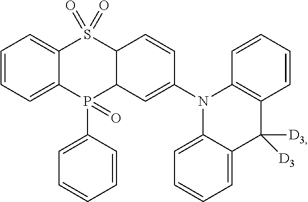

an emission wavelength of the electroluminescent material with the molecular structural formula

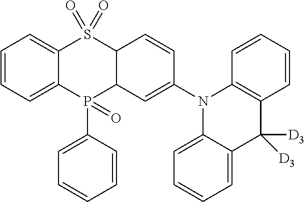

ranges from 450 nanometers to 475 nanometers.

Referring to Table 1, table 1 is a peak value of a fluorescence spectrum of an electroluminescent material and an energy level value of each energy state of the electroluminescent material.

| | PL | | | | | |
|---|---|---|---|---|---|---|
| | Peak | S₁ | T₁ | ΔE_{ST} | HOMO | LUMO |
| | (nm) | (eV) | (eV) | (eV) | (eV) | (eV) |
| 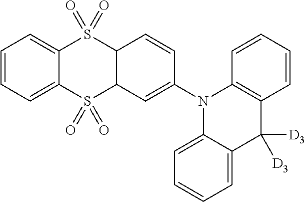 | 436 | 2.85 | 2.78 | 0.07 | −5.55 | −2.43 |
| 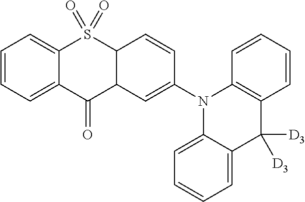 | 449 | 2.76 | 2.66 | 0.10 | −5.69 | −2.44 |
| 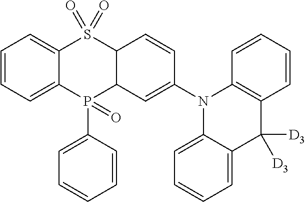 | 463 | 2.68 | 2.53 | 0.15 | −5.61 | −2.43 |

Wherein, PL Peak is a peak value of the fluorescence spectrum of the electroluminescent material, S1 is the lowest singlet energy level value, T1 is the lowest triplet energy level value, ΔE_{ST}=S1−T1, HOMO is the highest electron occupied orbit value, LUMO is the lowest electron unoccupied orbit value.

The present application provides a method for manufacturing an electroluminescent material. The method including:

A first reactant and a second reactant are provided. The first reactant and the second reactant are reacted to generate the electroluminescent material. A molecular structural formula of the first reactant is

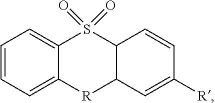

### and the R is

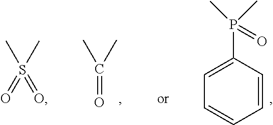

the R′ is Cl, Br, or I, the second reactant is 9, 10-dihydrogen-9, 9-dideuterium methyl acridine, a molecular structural formula of the second reactant is

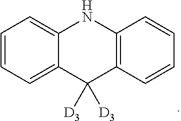

In one embodiment, a mole ratio of the first reactant and the second reactant ranges from 1:0.5 to 1:2. In other embodiments, the mole ratio of the first reactant and the second reactant equals to 1:0.8, 1:1, or 1:1.5 etc. In other embodiments, a relationship between the first reactant and the second reactant is that for 10 millimoles of the first reactant, there are 9 millimoles of the second reactant, that for 2 millimoles of the first reactant, there are 2.5 millimoles of the second reactant, or that for 50 millimoles of the first reactant, there are 60 millimoles of the second reactant.

In one embodiment, the first reactant and the second reactant are reacted in a solvent to generate an electroluminescent material. The solvent is selected from a group consisting of styrene, perchloroethylene, methylbenzene, trichloroethylene, acetone, ethylene glycol ether, and triethanolamine. The solvent includes an additive, and the additive is selected from a group consisting of potassium hydroxide, sodium hydroxide, sodium hydrogencarbonate, [1,1′-bis (diphenylphosphino) ferrocene] palladium dichloride (Pd(dppf)Cl₂), potassium acetate (KOAc), sodium acetate (NaOAc), potassium nitrate (KNO₃), palladium acetate (Pd(OAc)₂), magnesium sulfate (MgSO₄), sodium t-butoxide (NaOt-Bu) and tri-tert-butylphosphine tetrafluoroborate.

In one embodiment, a reaction temperature of the first reactant and the second reactant reacting to generate the electroluminescent material ranges from 100 degrees centigrade to 150 degrees centigrade. A reaction time of the first reactant and the second reactant reacting to generate the electroluminescent material ranges from 12 hours to 64 hours. The mixture containing the electroluminescent material is separated and purified to obtain the electroluminescent material by employing a group consisting of dichloromethane, trichloromethane, and tetrahydrofuran.

In one embodiment, when the R is

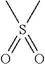

and the R′ is Br, a molecular structural formula of the first reactant is

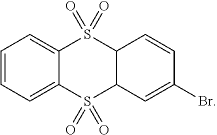 [SMILES: O=S1(=O)c2ccccc2S(=O)(=O)C2C=C(Br)C=CC21]

A reaction formula of the first reactant and the second reactant reacting to generate the electroluminescent material is:

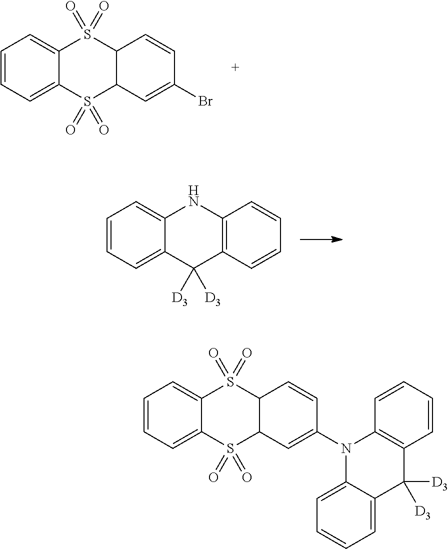

In one embodiment, 10 millimoles of the

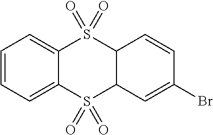 [SMILES: O=S1(=O)c2ccccc2S(=O)(=O)C2C=C(Br)C=CC21]

are added to a 200 mL two-neck flask, 15 millimoles of the 9, 10-dihydrogen-9, 9-dideuterium methyl acridine, 0.2 millimoles of the palladium acetate and 1 millimoles of the tertiary butyl phosphorus tetrafluoroborate are added, and then 15 millimoles of the sodium tert-butoxide (NaOt-Bu) are added in a glove box, and 40 mL of the methylbenzene are added in an argon atmosphere, the abovementioned are reacted 30 hours in 110 degrees centigrade to obtain a mixture, and the mixture is separated and purified to obtain the electroluminescent material

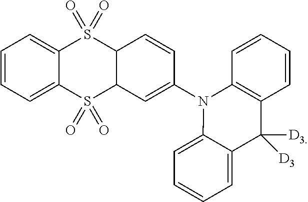

In one embodiment, when the R is

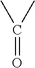

and the R′ is Br, a molecular structural formula of the first reactant is

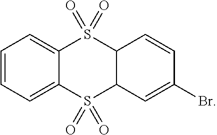 [SMILES: O=S1(=O)c2ccccc2S(=O)(=O)C2C=C(Br)C=CC21]

A reaction formula of the first reactant and the second reactant reacting to generate the electroluminescent material is:

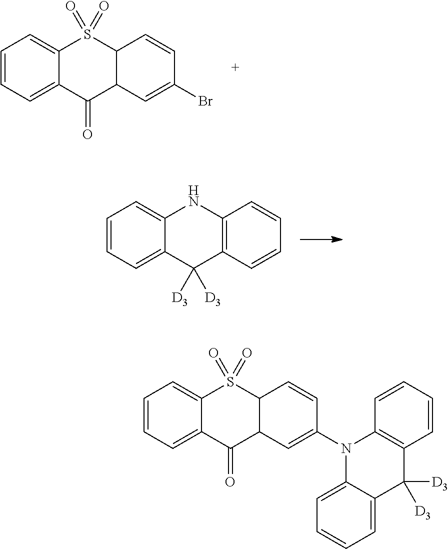

In one embodiment, 5 millimoles of the

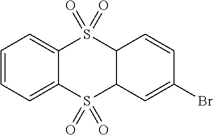 [SMILES: O=S1(=O)c2ccccc2S(=O)(=O)C2C=C(Br)C=CC21]

are added to a 200 mL two-neck flask, 4 millimoles of the 9, 10-dihydrogen-9, 9-dideuterium methyl acridine, 0.4 millimoles of the palladium acetate and 1 millimoles of the tertiary butyl phosphorus tetrafluoroborate are added, and then 15 millimoles of the sodium tert-butoxide (NaOt-Bu) are added in a glove box, and 40 mL of the methylbenzene are added in an argon atmosphere, the abovementioned are reacted 36 hours in 120 degrees centigrade to obtain a mixture, the mixture is separated and purified to obtain the electroluminescent material

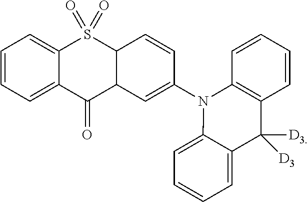

In one embodiment, when the R is

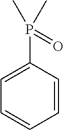

and the R′ is Br, a molecular structural formula of the first reactant is

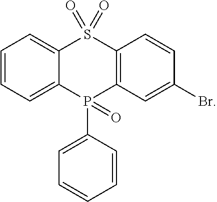 [SMILES: O=P1(c2ccccc2)c2ccccc2S(=O)(=O)c2ccc(Br)cc21]

A reaction formula of the first reactant and the second reactant reacting to generate the electroluminescent material is:

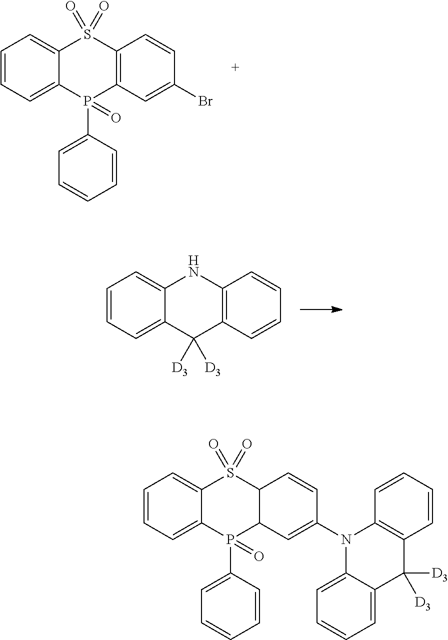

In one embodiment, 3 millimoles of the

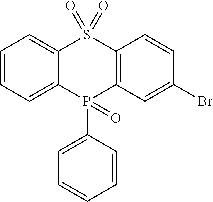 [SMILES: O=P1(c2ccccc2)c2ccccc2S(=O)(=O)c2ccc(Br)cc21]

are added to a 200 mL two-neck flask, 4 millimoles of the 9, 10-dihydrogen-9, 9-dideuterium methyl acridine, 0.1 millimoles of the palladium acetate and 0.5 millimoles of the tertiary butyl phosphorus tetrafluoroborate are added, and then 10 millimoles of the sodium tert-butoxide (NaOt-Bu) are added in a glove box, and 40 mL of the methylbenzene are added in an argon atmosphere, the abovementioned are reacted 48 hours in 110 degrees centigrade to obtain a mixture, the mixture is separated and purified to obtain the electroluminescent material

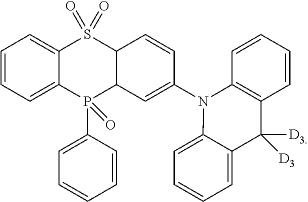

In a separating and purifying process, a reaction solution is cooled down to a room temperature, then the reaction solution is poured into ice water and extracted two to five times by an extraction solvent to obtain an organic phase, the organic phase are spinned into a silica gel, and the silica gel are employed column chromatography by a chromatographic column to obtain a white powder which is also defined as an electroluminescent material. The yield of the electroluminescent material is greater than or equal to 55%.

The extraction solvent is selected from a group consisting of dichloromethane, trichloromethane, and tetrahydrofuran. A volume ratio of dichloromethane and n-hexane of the chromatographic column ranges from 1:0.5 to 1:10.

Referring to FIG. 2, FIG. 2 is a structural view of a luminescent device of the present application.

A luminescent device 10 is provided. The luminescent device 10 includes a base layer 11, a cavity transmitting and injecting layer 12, a luminescent layer 13, an electronic transmitting layer 14, and a second electrode layer 15.

The base layer 11 includes a substrate base 111 and a first electrode layer 112 formed on the substrate base 111. The cavity transmitting and injecting layer 12 is formed on the base layer 11. The cavity transmitting and injecting layer 12 is electrically connected to the first electrode layer 112. The electronic transmitting layer 14 is formed on the luminescent layer 13. The second electrode layer 15 is electrically connected to the electronic transmitting layer 14.

The substrate base 111 is made of a glass substrate. The first electrode layer 112 is made of indium tin oxide. The cavity transmitting and injecting layer 12 is made of 3,4-ethylenedioxythiophene: polystyrene sulfonates (PEDOT:PSS). The electronic transmitting layer 14 is made of 1,3,5-tri(3-(3-pyridine)phenyl) benzene (Tm3PyPB). The second electrode layer 15 is made of lithium fluoride/aluminum (LiF/Al). The luminescent layer 13 includes an electroluminescent material, a molecular structural formula of the electroluminescent material is:

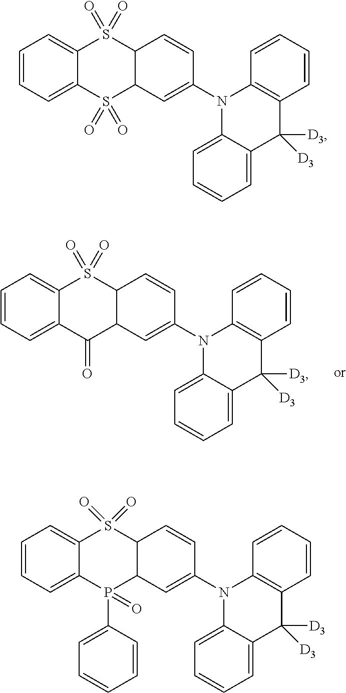

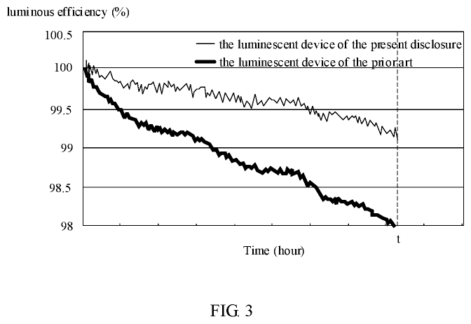

Referring to FIG. 3, FIG. 3 is a comparison chart of a luminous efficiency attenuated by time of luminescent devices of the present application and prior art. Referring to Table 2, table 2 shows performance data of luminescent devices of the present application and prior art.

TABLE 2

| | | | maximum |
|---|---|---|---|
| | | | external |
| | | chromaticity | quantum |
| | maximum current | coordinate | efficiency |
| | efficiency (cd/A) | CIEy | (%) |
| luminescent device of | 15.3 | 0.09 | 20.7 |
| prior art | | | |
| luminescent device of | 15.4 | 0.09 | 20.8 |
| the present application | | | |

The present application provides a luminescent device having a comparable maximum current efficiency, a same chromaticity coordinate, and a comparable maximum external quantum efficiency with a prior luminescent device, and the luminescent device of the present application provided shows that the luminescent efficiency attenuation percentage is less than 1% at time t, but the luminescent efficiency attenuation percentage of the luminescent device of prior art is 2% at time t.

An electroluminescent material, a method for manufacturing the electroluminescent material and a luminescent device are provide d, the method includes the following steps: providing a first reactant and a second reactant, reacting the first reactant and the second reactant to generate a mixture containing the electroluminescent material, and a molecular structural formula of the first reactant is

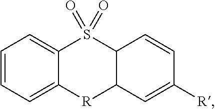

### and the R is

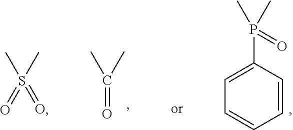

the R′ is Cl, Br, or I, the second reactant is 9, 10-dihydrogen-9, 9-dideuterium methyl acridine, a molecular structural formula of the second reactant is

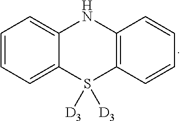

separating and purifying the mixture containing the electroluminescent material to obtain the electroluminescent material to generate a blue light emitting electroluminescent material with a long lifespan, and a method for manufacturing the electroluminescent material, and a luminescent device.

As is understood by a person skilled in the art, the foregoing preferred embodiments of the present disclosure are illustrative rather than limiting of the present disclosure. It is intended that they cover various modifications and similar arrangements be included within the spirit and scope of the present disclosure, the scope of which should be accorded the broadest interpretation so as to encompass all such modifications and similar structures.

What is claimed is:

1. An electroluminescent material, wherein a molecular structural formula of the electroluminescent material is as follows: 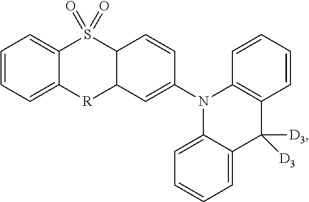
wherein R is

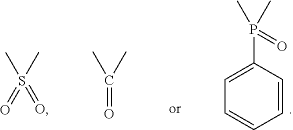

2. The electroluminescent material of claim 1, wherein an emission wavelength of the electroluminescent material ranges from 425 nanometers to 475 nanometers.

3. The electroluminescent material of claim 2, wherein the R is 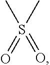
a molecular structural formula of the electroluminescent material is 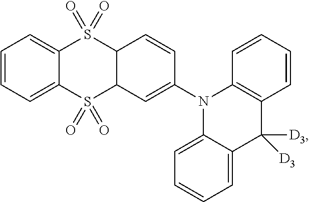
an emission wavelength of the electroluminescent material with the molecular structural formula 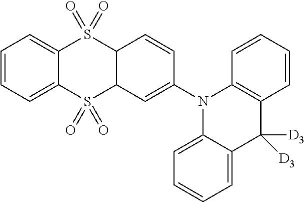
ranges from 425 nanometers to 450 nanometers.

4. The electroluminescent material of claim 2, wherein the R is 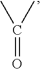
a molecular structural formula of the electroluminescent material is 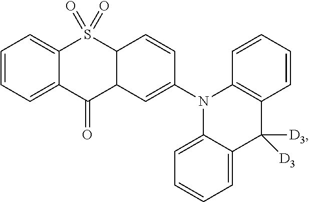
an emission wavelength of the electroluminescent material with the molecular structural formula 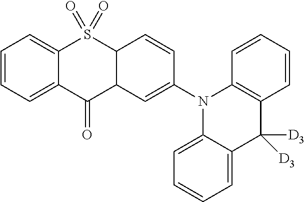
ranges from 430 nanometers to 460 nanometers.

5. The electroluminescent material of claim 2, wherein the R is 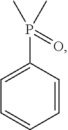
a molecular structural formula of the electroluminescent material is 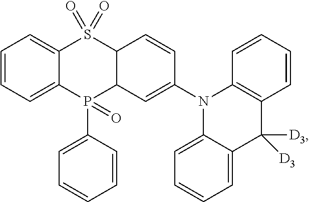
an emission wavelength of the electroluminescent material with the molecular structural formula 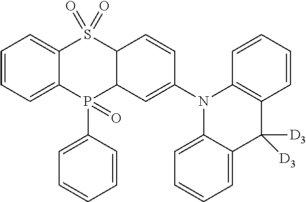
ranges from 450 nanometers to 475 nanometers.

6. A method for manufacturing an electroluminescent material, wherein comprising:
providing a first reactant and a second reactant, reacting the first reactant and the second reactant to generate the electroluminescent material, wherein a molecular structural formula of the first reactant is 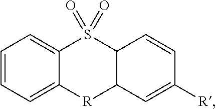
and the R is 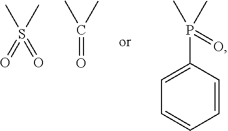
the R′ is Cl, Br, or I, the second reactant is 9,10-dihydrogen-9,9-dideuterium methyl acridine, and a molecular structural formula of the second reactant is

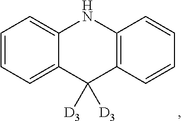

7. The method for manufacturing the electroluminescent material of claim 6, a mole ratio of the first reactant and the second reactant ranges from 1:0.5 to 1:2.

8. The method for manufacturing the electroluminescent material of claim 6, wherein the first reactant and the second reactant are reacted in a solvent to generate the electroluminescent material, and the solvent is selected from a group consisting of styrene, perchloroethylene, methylbenzene, trichloroethylene, acetone, ethylene glycol ether, and triethanolamine.

9. The method for manufacturing the electroluminescent material of claim 6, wherein the solvent comprises an additive, and the additive is selected from a group consisting of potassium hydroxide, sodium hydroxide, sodium hydrogencarbonate, [1,1′-bis(diphenylphosphino)ferrocene]palladium dichloride (Pd(dppf)Cl₂), potassium acetate (KOAc), sodium acetate (NaOAc), potassium nitrate (KNO₃), palladium acetate (Pd(OAc)₂), magnesium sulfate (MgSO₄), sodium t-butoxide (NaOt-Bu) and tri-tert-butylphosphine tetrafluoroborate.

10. The method for manufacturing the electroluminescent material of claim 6, wherein a reaction temperature of the first reactant and the second reactant reacting to generate the electroluminescent material ranges from 100 degrees centigrade to 150 degrees centigrade.

11. The method for manufacturing the electroluminescent material of claim 6, wherein a reaction time of the first reactant and the second reactant reacting to generate the electroluminescent material ranges from 12 hours to 64 hours.

12. The method for manufacturing the electroluminescent material of claim 6, wherein a molecular structural formula of the electroluminescent material is as follows: 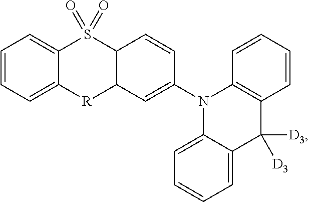
wherein R is

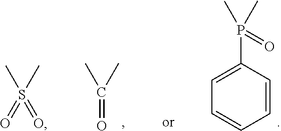

13. The method for manufacturing the electroluminescent material of claim 6, wherein the step of providing the first reactant and the second reactant, the first reactant and the second reactant are reacted to generate the electroluminescent material comprises:
providing a first reactant and a second reactant;
reacting the first reactant and the second reactant to generate a mixture containing the electroluminescent material; and
separating and purifying the mixture containing the electroluminescent material to obtain the electroluminescent material.

14. The method for manufacturing the electroluminescent material of claim 13, wherein the step of separating and purifying the mixture containing the electroluminescent material to obtain the electroluminescent material comprises:
extracting the mixture containing the electroluminescent material by an extraction solvent; and
employing column chromatography to the mixture containing the electroluminescent material by a chromatographic column.

15. The method for manufacturing the electroluminescent material of claim 14, wherein the extraction solvent is selected from a group consisting of dichloromethane, trichloromethane and tetrahydrofuran.

16. The method for manufacturing the electroluminescent material of claim 14, a volume ratio of the dichloromethane and n-hexane of the chromatographic column ranges from 1:0.5 to 1:10.

17. A luminescent device, wherein comprising:
a base layer, the base layer comprises a substrate base and a first electrode layer formed on the substrate base;
a cavity transmitting and injecting layer, the cavity transmitting and injecting layer is formed on the base layer, and electrically connected to the first electrode layer;
a luminescent layer, the luminescent layer is formed on the cavity transmitting and injecting layer;
an electronic transmitting layer, the electronic transmitting layer is formed on the luminescent layer; and
a second electrode layer, the second electrode layer is electrically connected to the electronic transmitting layer, wherein the luminescent layer comprises a electroluminescent material, a molecular structural formula of the electroluminescent material is: 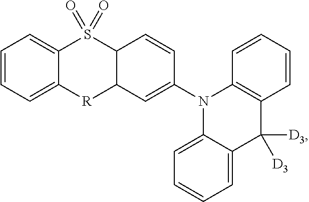
wherein the R is

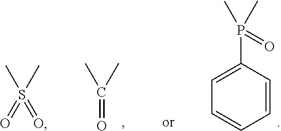

18. The luminescent device of claim 17, wherein the R is 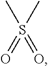
a molecular structural formula of the electroluminescent material is 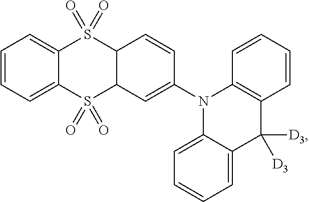
an emission wavelength of the electroluminescent material with the molecular structural formula 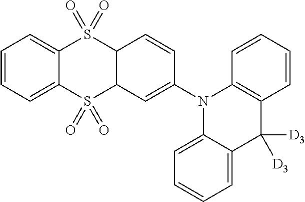
ranges from 425 nanometers to 450 nanometers.

19. The luminescent device of claim 17, wherein the R is 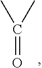
a molecular structural formula of the electroluminescent material is 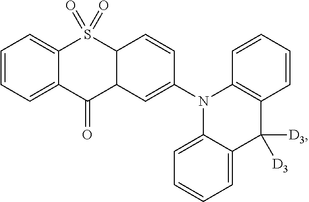
an emission wavelength of the electroluminescent material with the molecular structural formula 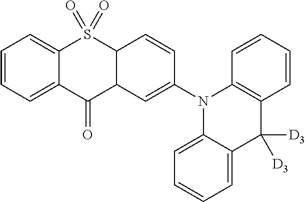
ranges from 430 nanometers to 460 nanometers.

20. The luminescent device of claim 2, wherein the R is 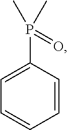
a molecular structural formula of the electroluminescent material is 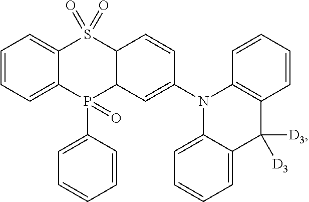
an emission wavelength of the electroluminescent material with the molecular structural formula 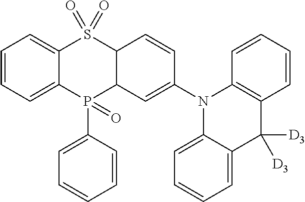
ranges from 450 nanometers to 475 nanometers.
